(19) United States
(12) Patent Application Publication
(10) Pub. No.: US 2005/0084908 A1
(43) Pub. Date: Apr. 21, 2005

(54) Methods for detecting binding of low-molecular-weight compound and its binding partner molecule

(75) Inventor: Keiko Esaki, Shizuoka (JP)

Correspondence Address: FOLEY AND LARDNER;SUITE 500, 3000 K STREET NW, WASHINGTON, DC, 20007, US

(73) Assignee: CHUGAI SEIYAKU KABUSHIKI KAISHA

(21) Appl. No.: 10/924,196

(22) Filed: Aug. 24, 2004

Related U.S. Application Data
(63) Continuation-in-part of application No. 09/783,391, filed on Feb. 15, 2001.
(63) Continuation-in-part of application No. PCT/JP03/02044, filed on Feb. 25, 2003.
(60) Provisional application No. 60/245,560, filed on Nov. 6, 2000.

(30) Foreign Application Priority Data
Feb. 25, 2002 (JP) 2002-48450

(51) Int. Cl. C12Q 1/68; G01N 33/53; C12M 1/34

(52) U.S. Cl. 435/7.1; 435/287.2

(57) ABSTRACT

A method for detecting the binding between a binding molecule and an immobilized low-molecular-weight compound is provided. The method comprises a step of measuring volume changes due to the binding of both compounds as an indicator. The use of immobilized low-molecular-weight compound produces highly reliable measuring results in terms of surface plasmon resonance, etc. The detection method of this invention is useful for screening for low-molecular-weight compounds that bind to binding molecules, or binding molecules that bind to low-molecular-weight compounds.

### CROSS-REFERENCE TO RELATED APPLICATION

This application is a continuation-in-part of U.S. application Ser. No. 09/783,391, filed Feb. 15, 2001, which claims the benefit of U.S. application Ser. No. 60/245,560, filed Nov. 6, 2000. This application also claims priority from International Patent Application No. PCT/J03/02044, filed Feb. 25, 2003, which claims priority from Japanese Patent Application No. 2002-48450, filed Feb. 25, 2002.

### FIELD OF THE INVENTION

The present invention relates to a method for detecting the complex formation of a low-molecular-weight compound with its binding partner molecule, and a method for screening a novel binding partner molecule for known low-molecular-weight compounds as well as novel low-molecular-weight compounds capable of binding to targeting molecules using the method.

### BACKGROUND OF THE INVENTION

There have been hitherto developed a variety of methods and apparatuses for analyzing the protein-protein interaction. Such methods include affinity chromatography and immunoprecipitation using antibodies. In addition, there are methods for detecting changes in the distribution coefficient, buoyant density, electrophoretic mobility, absorbiance, etc. when molecules associate among themselves to form a large complex. A method using radioisotope developed by Obourn et al., in particular, is a technique that has been widely used, in which a tracer labeled with radioisotope and a substance to be assayed are competitively reacted with a receptor to measure the level of radioisotope in the tracer bound to the receptor (Obourn, J. D., et al., (1993) Biochemistry 32, 6229-6236).

Recently, much attention has been focused on a method for measuring protein-protein interaction using surface plasmon resonance (SPR) (Ward, L. D., et al., (1994) Journal of Biological Chemistry, 269, 23286-23289, Esaki, K et al., BIAsymposium '99, Abstracts for presentation; Kempter, et al., Analytica Chimica Acta 362 (1998) 101-111). Some apparatuses to measure SPR have been already developed and on the market.

To measure protein-protein interaction by SPR, one of the objective proteins is immobilized on the surface of a sensor chip, and a test sample containing a protein molecule that reacts with the protein is contacted with the sensor chip at a constant flow rate through a microfluidic system. As a complex is formed by the binding of the immobilized molecule to a test substance contained in the test sample, the volume of proteins on the sensor chip is increased. This increase in the volume of proteins on the sensor chip is optically detected in terms of SPR and represented in a graph called sensorgram. In addition, volume increases due to the complex formation among proteins can be converted into weight increases on the sensor chip. The intensity of SPR is known to correlate with weight changes. Differing from conventional methods, a real-time detection of molecular interactions by measuring SPR is advantageous in that a small amount of samples can be assayed in a short time without labeling reactants.

However, since SPR detects minute volume changes on sensor chips, a test substance preferably has a certain molecular weight, and it is difficult to detect the binding of a low-molecular-weight compound used as a test substance to an immobilized protein. When a compound of a molecular weight of 1,000 or less is used as a test compound, it is difficult to obtain reproducible and reliable results. When a compound of a molecular weight of 500 or less is used as a test compound, the change in SPR itself is extremely difficult to detect.

As can be seen in hormone and its receptor, ligand and its nuclear receptor, etc., many low-molecular-weight compounds play a critical role in the signal transduction in the living body. Therefore, it is highly significant to apply technology such as SPR measurement, which can analyze the binding between substances using a small amount of a sample in a short time, to analyses of the binding of low-molecular-weight compounds.

The conventional measurement method has problems that it is difficult to reuse a protein immobilized on the surface of a sensor chip because its conformation is altered by washing the sensor chip under severe conditions.

### SUMMARY OF THE INVENTION

The present inventors intensively studied an assay method simultaneously using multiple samples and reagents and have found that surface plasmon resonance of low-molecular-weight compounds immobilized on the surface of a carrier can be efficiently measured and that this method can be applied to screening of drugs. Furthermore, this method can complete the measurement in a short time, which minimizes the inactivation of the binding activity of a protein used as a test substance and provides reliable experimental data as compared with the conventional binding activity assay using radioisotopes.

One embodiment of this invention is a method for detecting the binding between an immobilized low-molecular-weight compound and a binding molecule. The method comprises the steps of (1) contacting the immobilized low-molecular-weight compound with the binding molecule to form a complex, and (2) measuring a volume change due to the binding between the low-molecular-weight compound and the binding molecule.

Another embodiment of this invention is a method for measuring the content of a binding molecule or a low-molecular-weight compound contained in a sample using the above method.

Still another embodiment of this invention is a method for screening a low-molecular-weight compound capable of binding to a target molecule or a binding molecule capable of binding to a known low-molecular-weight compound using the above method.

Yet another embodiment of this invention is a compound obtainable by the above screening method.

### BRIEF DESCRIPTION OF THE DRAWINGS

FIG. 1 represents the structures of 7α-(9-aminononyl)estradiol (4) and synthetic intermediates thereof.

FIG. 2 schematically illustrates the binding of 7α-(9-aminononyl)estradiol immobilized on the Sensor Chip CM5 and a binding molecule. The immobilized 7α-(9-aminononyl)estradiol is linked to the carboxyl group of carboxymethyl dextran on the sensor chip via the amino group inserted as the side chain. With this immobilized 7α-(9-aminononyl)estradiol is reacted with the ligand binding domain of human estrogen receptor-α (ERLBD) or a fusion protein of this domain and maltose binding protein (MBP-ERLBD), and volume changes due to the complex formation are real time displayed as a graph called sensorgram.

FIG. 3 is a sensorgram illustrating the binding of MBP-ERLBD or ERLBD to the immobilized 7α-(9-aminononyl)estradiol. When MBP-ERLBD (20 μg/ml, 10 μl), or ERLBD (20 μl, 10 μl) was injected (2 min), the binding corresponding to about 2000 resonance units (RU) was detected. At the time indicated by [Reg.] in the figure, 10 μl of 7% propanol/50 mM hydrochloric acid were injected (2 min) to regenerate and wash the sensor chip.

FIG. 4 is a bar graph demonstrating that the binding reaction of ERLBD to immobilized 7μ-(9-aminononyl)estradiol is a specific binding reaction when β-estradiol was used as the competitive reaction substance. A sample prepared by reacting ERLBD (20 μg/ml or 0.645 μM) and β-estradiol (363 μM; about 500 times molar concentration of ERLBD, SIGMA) for 1 hr at room temperature and ERLBD alone were examined for their binding activity to immobilized 7α-(9-aminononyl)estradiol. An excessive amount of β-estradiol almost completely inhibited the binding of ERLBD to immobilized 7α-(9-aminononyl)estradiol.

FIG. 5 schematically illustrates a conventional binding assay using a ligand labeled with radioisotope (RI). ³H-ligand stands for [6,7-³H]estradiol, MBP-LBD for MBP-ERLBD, and LBD for ERLBD After the ligand and the binding molecule are mixed and allowed to stand at room temperature for 6 hr, the resulting mixture is subjected to the Bound/Free separation procedure. The amount of [6,7-³H]estradiol bound to MBP-ERLBD or ERLBD is detected with a scintilator.

FIG. 6 is a bar graph showing the comparison of binding activity assayed by BIACORE method of this invention and that assayed by the RI method. The binding activity of MBP-ERLBD was detected by both the BIACORE and RI methods. In contrast, the binding activity of ERLBD was detected only by the BIACORE method, and the RI method detected only about 10% of the expected binding activity.

FIG. 7 shows the inhibitory activities of various concentrations of the test compounds against binding of immobilized (1α,3β,5Z,7E)-25-(10-aminodecanyl)-27-nor-9,10-secocholesta-5,7,10(19)-triene-1,3,25-triol (hereinafter, referred to as ED-533) to a vitamin D3 receptor (VDR). The percent inhibition is shown on the vertical axis, and the concentration of each test compound is shown on the horizontal axis.

FIG. 8 shows the scheme of ED-533 synthesis.

### DETAILED DESCRIPTION OF THE INVENTION

SPR can be employed to measure volume changes due to the formation of a complex of an immobilized low-molecular-weight compound and a substance to be tested or a targeting molecule. Commercially available products, for example, BIACORE (Biacore) or IBIS (Intersens) system, can be used. SPR and general experimental methods using BIACORE can be referred to a reference “Real-Time Analysis of Biomolecular Interaction: Application of BIACORE” Springer, Nagata, K, and Handa, H. eds. (2000) (which is incorporated herein by reference). Herein, a volume change can be a change of weight, density, or concentration. Therefore, a volume change is synonymous with a weight change, a density change, or a concentration change.

One embodiment of this invention relates to a method for measuring the binding activity of an immobilized low-molecular-weight compound to a test substance (binding molecule) in a test sample. There is no particular limitation on low-molecular-weight compounds to be immobilized on the surface of sensor chips, which may be naturally occurring molecules or artificially synthesized ones. Naturally occurring molecules include those present in living bodies such as humans and mammals, and those produced by plants and microorganisms. Any type of low-molecular-weight compounds, including organic compounds, peptides, DNA fragments, etc., can be employed. The molecular weight of low-molecular-weight compounds is preferably in the range of 50 to 5,000, more preferably 50 to 2,000, and further preferably 50 to 1,000, and most preferably 50 to 500.

For example, when low-molecular-weight compounds are peptides, the number of amino acid residues is preferably 2 to 50, more preferably 20 or less, further more preferably 10 or less, and most preferably 5 or less. Amino acids may be naturally occurring amino acids, optical isomers thereof, or modified amino acids with altered side chains. The peptides may be cyclic peptides. Organic compounds means compounds constituted of mainly carbon atoms and hydrogen atoms, and optionally nitrogen atoms, sulfur atoms, halogen atoms, etc., and may be organometallic compounds with metallic atom. Therefore, in a broad sense, amino acids and nucleic acids are also included in organic compounds.

Low-molecular-weight compounds with physiological or biological activities which are present in the living body include, for example, adrenocortical hormones, physiologically or biologically active lipids, and neurotransmitters. Specific examples are glucocorticoids, mineralocorticoids, vitamin D₃ and its derivatives, female hormones, male hormones, thyroid hormones, vitamin A, prostaglandins, leukotriens, arachidonic acid, etc. More specifically, estrogens (estradiol, estrone, and estriol), androgens (testosterone and dihydrotestosterone), 1,25-hydroxylated vitamin D₃, adrenaline, noradrenaline, histamine, dopamine, serotonin, progesterone, cortisol, corticosterone, aldosterone, thyroxine, retinol, retinal, retinoic acid, prostaglandin E₂, leukotriene B₄, etc. can be used.

Existing drugs can be used as the low-molecular-weight compounds with physiological activities which are not present in the living body. Structural and functional analogues of such low-molecular-weight compounds with physiological or biological activity can also be used. Since there have been a great number of reports on these structural and functional analogues, those skilled in the arts can easily search, prepare, or obtain them. For example, synthetic estrogen analogues such as diethylstilbestrol, hexestrol, etc. can be used.

A low-molecular-weight compounds can be immobilized on a carrier such as a sensor chip or cuvette for SPR, directly by the covalent bond or indirectly. The indirect immobilization may be carried out using two kinds of molecules that are known to bind to each other; one molecule may be immobilized on the carrier surface, and the other molecule may be linked to a low-molecular-weight compound which is to be immobilized. For example, a combination of biotin-avidin or a suitable antigen and its antibody may be employed.

A low-molecular-weight compound can be immobilized on the sensor chip surface by a covalent bond by directly linking the compound to a carboxymethyl group introduced to the dextran layer coated on the sensor chip. For example, carboxyl groups of carboxymethyl groups can be activated by a mixture of N-ethyl-N′-(3-dimethyl-aminopropyl)carbodiimide hydrochloride (EDC) and N-hydroxysuccinimide (NHS), and covalently bound to functional groups such as amino group, thiol group, hydroxyl group, etc. of the low-molecular-weight compound. Carboxyl groups can be covalently bound to aldehyde groups mediated by hydrazine. When a low-molecular-weight compound has no suitable functional group, a functional group can be introduced at suitable sites. Reaction conditions, solvents, and the like required for the covalent binding can be suitably altered as the occasion demands.

A carboxymethyl group and a low-molecular-weight compound may be covalently bound via a linker with an appropriate chain length. Any linker molecules may be used as long as they have appropriate functional groups at both ends. The distance between the two functional groups at both ends is, in terms of the carbon chain, preferably 50 atoms or less, more preferably 30 atoms or less, further more preferably 20 atoms or less, and most preferably 10 atoms or less. Preferably, linker molecules are not substantially involved in the immobilization of low-molecular-weight compounds or the binding between immobilized low-molecular-weight compounds and substances to be tested, and are chemically stable against various solvents. For example, such linker molecules include substituted or unsubstituted alkyl or alkenyl chains having functional groups necessary for covalent binding on both ends. Suitable linkers can be selected and prepared by techniques well known to those skilled in the art. For the stable immobilization of low-molecular-weight compounds, the linkers are preferably immobilized on sensor chips directly by a covalent bond.

There is no particular limitation on binding partner molecules to be assayed for their binding activities to immobilized low-molecular-weight compounds as long as they have a molecular weight necessary for the detection of changes in SPR signals. Any desired molecules can be used as test substances. The binding molecules may be, for example, proteins, single-stranded DNA, double-stranded DNA, sugar chains, non-peptidic compounds, organic compounds, etc. The molecular weight necessary for the detection of changes in SPR is the one that produces a detectable level of changes in SPR signals, which are caused by formation of a complex of a test substance and an immobilized low-molecular-weight compound, and is preferably 2 kD or more, more preferably 5 kD or more, further more preferably 10 kD or more, and most preferably 30 kD or more. The detectable level of changes in SPR signals is 10 resonance unit (10 pg/mm²), and, as the reliable signal, preferably 50 resonance unit (50 pg/mm²).

Binding molecules are preferably purified to measure the binding activity of immobilized low-molecular-weight compounds to the binding molecules. Therefore, recombinant proteins, which can be easily purified, are preferred binding molecules. Recombinant proteins can be prepared by known methods using a combination of a suitable vector and host cells (Molecular Cloning 2nd Edt., Cold Spring Harbor Laboratory Press, 1989; Basic Methods in Molecular Biology, Appleton & Lange, 1986). Recombinant proteins and naturally occurring proteins can be isolated and purified by methods for separating and purifying usual proteins without any limitations.

For example, affinity chromatography, ion-exchange chromatography, hydrophobic chromatography, gel filtration chromatography, reverse phase chromatography, filtration, salting-out, immunoprecipitation, electrophoresis, etc. can be used alone or in any suitable combinations (Strategies for Protein Purification and Characterization: A Laboratory Course Manual, Ed. Daniel R. Marshak et al., Cold Spring Harbor Laboratory Press, 1996).

When a compound having a too small molecular weight as the binding molecule is used or only a specific partial amino acid sequence of a protein is used to exclude non-specific bindings, marker molecules can be linked to these compounds so that the binding molecules can have suitable molecular weights necessary for detecting changes in SPR signals. There is no limitation on marker molecules as long as they have suitable molecular weights. They may be proteins, DNAs, macromolecular organic compounds, or beads. Marker molecules are preferably hydrophilic.

When a protein is used as a marker molecule, its fusion protein with a binding molecule may be prepared using known genetic engineering techniques by linking DNA encoding a substance to be tested (binding molecule) and DNA encoding a marker molecule so that reading frames are matched, introducing the resulting recombinant DNA into an expression vector, and allowing the DNA to be expressed in appropriate host cells. A cleavage site of a suitable enzyme may be inserted between the marker molecule and the substance to be tested beforehand, so that the marker molecule and the targeting molecule can be cleaved as the occasion demands. The enzyme cleavage sites may be, for example, those for Factor Xa, enterokinase, and genenase I. Expression vectors comprising marker molecules and the cleavage sites can also be used, and are available, for example from New England BioLabs.

An example of the marker molecules is, for example, maltose-binding protein (MBP). Macromolecular organic compounds that can be used include, for example, PEG, poly-lactic acid, etc. A marker molecule and a binding molecule can be bound using a linker. When beads are used as the marker molecule, they may be directly bound to the binding molecule, or the compound-immobilized beads synthesized using combinatorial chemical techniques may be used as the binding molecule.

When highly hydrophobic binding molecules are used, solutions containing organic solvents can be used as a solvent for a test substance. Although there is no particular limitation on organic solvents to be used, dimethyl sulfoxide (DMSO), methanol, ethanol, propanol, acetonitrile, etc. can be used at a concentration of preferably 1 to 8%, more preferably 1 to 3%.

Another embodiment of this invention relates to a method for measuring the content of a binding molecule in a sample, in which a low-molecular-weight compound that is known to bind to the binding molecule is immobilized. There is no particular limitation on samples, and tissues, supernatants of cell cultures, solubilized fractions, and fractions obtained at various purification procedures such as chromatography, etc. can be used as they are, or after appropriately concentrated or diluted as the occasion demands. For the dilution, the running buffer is preferably used.

The content of a binding molecule in a sample can be calculated as the intensity of SPR signals. The binding molecule content in samples can be easily compared using a calibration curve prepared beforehand using the purified binding molecule, but not essential. It is also possible to prepare test samples at different dilutions and compare the signal intensity at varied dilutions. The present method enables comparison of expression levels of binding molecules in each tissue, confirmation of the purification grade of binding molecules that are produced using genetic engineering techniques or isolated from natural sources, and identification of the active fractions.

The method can also be used to obtain the association and dissociation rates, and the association constant between a binding molecule and an immobilized low-molecular-weight compound. A binding molecule is serially diluted to obtain sensorgrams at each concentration. From sensorgrams thus obtained the reaction rate constant is calculated using the method of non-linear least squares regression, which can be easily calculated using an analysis software (BIAevaluation version 2.1, or 3.0) provided with BIACORE.

This invention enables measuring not only binding molecules but also low-molecular-weight compounds contained in test samples. That is, an immobilized low-molecular-weight compound and a low-molecular-weight compound present in test samples are competitively bound to a predetermined amount of a binding molecule to measure the low-molecular-weight compound present in test samples. The competitive reaction can be carried out by contacting a predetermined amount of the binding molecule with the immobilized low-molecular-weight compound in the presence of a test sample or contacting the binding molecule first with a test sample and then with the immobilized low-molecular-weight compound. As a result, the amount of the binding molecule to bind to the immobilized low-molecular-weight compound is reversely proportional to the concentration of the low-molecular-weight compound contained in a test sample.

Yet another embodiment of this invention relates to a method for detecting the binding activity of a low-molecular-weight compound to a binding molecule.

Furthermore, this invention relates to a method for screening a low-molecular-weight compound capable of binding to a targeting molecule. A low-molecular-weight compound capable of binding to a targeting molecule is useful as an agonist, antagonist, inhibitor, or stimulator of the targeting molecule. Such a low-molecular-weight compound is also useful as a ligand that can be used to detect or purify the targeting molecule. The “targeting moelcule” used herein means binding molecules used for screening low-molecular-weight compounds.

In this method, a low-molecular-weight compound as a test substance is immobilized and reacted with a targeting molecule to measure volume changes due to the formation of a complex resulted from the interreaction of the targeting molecule with the immobilized low-molecular-weight compound. In this case, the targeting molecule may be protein, DNA, or sugar. Examples of the proteins include blood proteins such as cytokines or lymphokines, cell-constituting proteins, cell membrane receptors, nuclear receptors, enzymes, ion channels, nuclear factors, transcription regulators, intracellular signal transducers, antibodies, and single-chain antibodies, etc. Orphan receptors can also be used (Kliewer, S. A. (1999) Science, 284, 757-760). Although these targeting molecules may be isolated and purified from natural sources, or produced by genetic engineering techniques, they are preferably purified as highly as possible, so that non-specific reactions can be eliminated as much as possible.

Furthermore, targeting molecules may be proteins, including full-length peptides or their specific region or part. For example, they may be purified from natural sources, or partial proteins obtained by the treatment with protease(s). Targeting molecules can be produced by genetic engineering techniques as a whole protein or only a specific partial sequence thereof. The specific region may be, for example, the ligand binding region and extracellular region of a receptor, the variable region of antibody, Fab fragment, single-chain antibody, etc.

A low-molecular-weight compound may be immobilized on the sensor chip surface, and reacted with a purified targeting molecule to measure the binding activity of the low-molecular-weight compound in terms of changes in SPR signals. Multiple targeting molecules can be conveniently measured by preparing two or more kinds of targeting molecules and reacting them with an immobilized low-molecular-weight compound one after another to measure changes in SPR signals. A mixture of multiple targeting molecules can also be measured.

After the complex formation is observed by changes in SPR signals, sensor chips are washed under appropriate conditions, and a targeting molecule bound to an immobilized low-molecular-weight compound may be recovered and analyzed by mass spectrometry or the like method. In this case, the immobilized low-molecular-weight compound is capable of binding to the recovered targeting molecule, indicating that the low-molecular weight compound is an agonist or stimulator, or an antagonist or inhibitor, for the molecule.

Another embodiment of this invention relates to a method for detecting compounds that bind to targeting molecules to interfere with the binding of targeting molecules to low-molecular-weight compounds that are known to bind to the targeting molecules. This method detects the binding activity of low-molecular-weight compounds to binding molecules, comprising the following steps:
(1) immobilizing a test low-molecular-weight compound or a known low-molecular-weight compound capable of binding to a binding molecule,
(2) forming a complex between the test low-molecular-weight compound and the binding molecule, wherein, when the known low-molecular-weight compound is immobilized, the complex is formed by any of the following methods (a) to (c),
   (a) contacting the known low-molecular-weight compound with the binding molecule in the presence of the test low-molecular-weight compound,
   (b) contacting the binding molecule with the test low-molecular-weight compound, and then with the known low-molecular-weight compound, and
   (c) contacting the binding molecule with the known low-molecular-weight compound, and then with the test low-molecular-weight compound, and
(3) measuring volume changes due to the binding of the immobilized low-molecular-weight compound to the binding molecule.

The method (a) in the step (2) measures the competition activity of a test low-molecular-weight compound against the binding reaction between a known low-molecular-weight compound and a targeting molecule. When the binding between the targeting molecule and the immobilized low-molecular-weight compound is not observed in the presence of the test substance, the test substance has the activity to bind to the targeting molecule, and can be identified as an agonist or stimulator, or an antagonist or inhibitor for the molecule.

The method (b) measures the activity of a test low-molecular-weight compound to inhibit the binding reaction between a known low-molecular-weight compound and a targeting molecule. When the test compound has the activity to bind to the targeting molecule, the binding of the targeting molecule and the known low-molecular-weight compound is inhibited.

The method (c) measures the replacement activity of a test low-molecular-weight compound for the binding reaction between a known low-molecular-weight compound and a targeting molecule. In this method, if the test compound has the activity to bind to the targeting molecule, a portion of the targeting molecule which has bound to the known low-molecular-weight compound is replaced with the test low-molecular-weight compound, resulting in a decrease in complex formation.

By any of these methods, the binding activity of a test compound to a targeting molecule can be detected. Test low-molecular-weight compounds can thus be screened by selecting those having the binding activity using this method. Low-molecular-weight compounds selected by this method are useful as agonists or stimulators, or antagonists or inhibitors, for the targeting molecules.

Whether compounds found by these methods which have the activity to bind to a targeting molecule are agonists or stimulators, or antagonists or inhibitors can be measured using well known methods, for example, by measuring the physiological activity of the low-molecular-weight compounds since a known targeting molecule and a known low-molecular-weight compound which binds to the targeting molecule are used in the method according to this invention. If the physiological activity of a targeting molecule is known, the physiological activity can be measured for this purpose.

For example, when the targeting molecule is a nuclear receptor, and it has been proved that the DNA sequence-specific transcription activity is enhanced by the binding of a low-molecular-weight compound, a ligand, to the receptor, the enhancement of transcription activity using a newly found compound, and the reduction of transcription activity by the competitive reaction with a ligand may be determined by measuring the transcription activity. When the enhancement of transcription activity is observed in the presence of a compound, the compound is proved to be an agonist, and when the transcription activity is reduced due to the binding of a ligand, the ligand is found to be an antagonist.

Changes in the transcription activity can be measured using a well-known method such as the reporter gene assay, etc. In the case of the cell membrane receptor molecule, changes in the physiological activity, in place of the transcription activity, induced by the stimulation of the cell membrane receptor may be observed. For example, it is possible to measure the auto-phosphorylation/dephosphorylation of the intracellular domain of receptors and phosphorylation/dephosphorylation of signal transmitters, and the proliferation activity of cells expressing the receptor.

When a targeting molecule is an enzyme, the enzyme activity may be assayed. It is proved that, when the enzyme activity is elevated in the presence of a compound, the compound is a stimulator for the enzyme, and when the inhibition of enzyme activity is induced, the compound is an inhibitor.

One of characteristics of the method in this invention is to use a combination of a targeting molecule and a low-molecular-weight compound that is known to bind to the targeting molecule. In many cases, physiological activities of these compounds are also known. Therefore, by measuring the known physiological activity, it can be determined whether a newly discovered compound capable of binding to the targeting molecule is an agonist or stimulator, or an antagonist or inhibitor.

There is no particular limitation on targeting molecules as long as low-molecular-weight compounds capable of binding to the targeting molecules are known, and receptors binding to low-molecular-weight compounds, the ligand, can be preferably used. Herein, receptors include cell membrane receptors, membrane receptors inside cells, and nuclear receptors within the nucleus.

Furthermore, targeting molecules besides receptors can be ion channels, enzymes, intracellular signal transmitters, etc. Ion channels can be used if low-molecular-weight compounds which bind to the ion channels and either stimulate or inhibit the action of ion channels are known.

Enzymes can be used if their substrates, competitive inhibitors, or allosteric inhibitors are known. Furthermore, intracellular signal transmitters can be used if low-molecular-weight compounds having the activity to stimulate or inhibit the enzymatic activity of the signal transmitter, for example, the phosphorylation or dephosphorylation activity are known.

Nuclear receptors and cell membrane receptors can be used as the receptor. Examples of nuclear receptors include androgen receptors, estrogen receptors, vitamin D₃ receptors, glucocorticoid receptors, mineralocorticoid receptors, progesterone receptors, thyroid hormone receptors, retinoic acid receptors, etc. Cell membrane receptors include cytokine receptors, lymphokine receptors, hematopoietic factor receptors, etc. As receptors for hematopoietic factors such as erythropoietin, granulocyte growth factor or granulocyte colony-stimulating factor(G-CSF), thrombopoietin, etc.; cytokines; lymphokines; etc., low-molecular-weight compounds having the activity to mimic or inhibit the action of natural proteinic ligands may be used to be immobilized (U.S. Pat. Nos. 6,107,304 and 5,981,551).

When the natural ligand is a low-molecular-weight compound, it can be immobilized. Alternatively, its structural and functional analogues can also be immobilized. Herein, functional analogues refer to compounds having similar biological activities as the above-described biologically active low-molecular-weight compounds. Functional analogues include any compound having similar activity as that of the natural ligand, regardless of whether its activity is strong or weak. Structural analogues used herein refer to compounds having various modifications made on the structure characteristic of a compound. Structural analogues may be artificially synthesized, or naturally occurring compounds. While functional analogues of a compound have similar activity as that of the compound, structural analogues do not necessarily have similar activity as that of the original compound. For example, diethylstilbestrol, hexestrol, or 7-α-(9-aminonoyl)estradiol may be used as estrogen receptors. Combinations of targeting molecules and low-molecular-weight compounds capable of binding to the targeting molecules can be referred to “Trends in Pharmacological Science: Receptor & Ion Channel Nomenclature Supplement, Alexander, S. P. H. and Peters, J. A. (2000).” A test substance and a targeting molecule may be mixed beforehand, and then examined for their binding to an immobilized low-molecular-weight compound, or after a targeting molecule is once bound to an immobilized low-molecular-weight compound, a test substance is reacted with the binding product to detect changes in SPR signals, the dissociation of the targeting molecule bound to the immobilized low-molecular-weight compound due to the replacement of it with a test.

Potent washing conditions can be used in the present method in which low-molecular-weight compounds are used as the immobilized molecule. In this invention, by washing sensor chips under appropriate washing conditions, it is possible to regenerate the sensor chip surface for its re-use without altering the structure of the immobilized low-molecular-weight compound. The washing solution includes, for example, 10 to 50% DMSO, 10 to 70% methanol, ethanol, and propanol, 0.01 to 0.1 M hydrochloric acid, 0.01 to 0.1 M sodium hydroxide solution, 8 M urea, surfactants such as 0.5% SDS solution, etc. and appropriate combinations thereof. In general, the higher the hydrophobicity, the ionic strength, and acidity or basicity of the washing solution is, the stronger the washing conditions need to be. The capacity of binding of targeting molecules to a large number of test substances can easily measured by repeating washing and reaction. Thus the present invention is extremely effective for screening drugs and identifying their physiological activities.

In another embodiment of this invention, unknown molecules capable of binding to low-molecular-weight compounds can be screened or identified. When changes in SPR signals are detected by reacting an immobilized low-molecular-weight compound with a test sample expected to contain a binding molecule, the test sample is found to be contain a binding molecule to the immobilized low-molecular-weight compound. A test sample may be a purified substance, or a mixture of two or more kinds of substances to be tested.

Furthermore, it is possible to use non-purified mixtures as the test sample. Such non-purified mixtures include, for example, supernatants of cell cultures or conditioned medium, cell extracts, and nuclear extracts. Cell culture supernatants containing recombinant proteins produced using animal cells, mammalian cells, Escherichia coli, insect cells, Bacillus subtilis, etc. or proteins purified from culture supernatants may be used as the test sample. A phage library can also be used as the test sample. In addition, Escherichia coli and non-adhering cells as they are can be used as the test sample. Cell extracts may be cell membrane fractions and intracellular fractions. The intracellular fractions can be soluble fractions. The cell membrane fractions can be solubilized with surfactants or detergent such as Nonidet P-40, sodium deoxycholate, etc.

When a mixture used as the test sample is reacted with an immobilized physiologically active low-molecular-weight compound to confirm the presence of a binding molecule in the mixture which binds to the immobilized physiologically active low-molecular-weight compound, the binding molecule can be recovered by successively washing sensor chips stepwise under from mild to strong washing conditions. The washing solutions contain molecules capable of binding to the immobilized low-molecular-weight compound. This method is very useful since the binding molecule can be recovered in washing solutions according to the binding intensity thereof. Such washing conditions can be those described above.

The method of this invention can also isolate and identify co-binding molecules besides binding molecules which directly bind to an immobilized low-molecular-weight compound. Co-binding molecules are also referred to as co-factors, having the capacity to regulate the activity of physiologically active or bioactive molecules. If the additional binding of the co-binding molecule to a complex between a low-molecular-weight compound and a binding molecule enhances physiological activity, the co-binding molecule is referred to as a co-factor. In contrast, when the activity is reduced, the co-binding molecule may be referred to as a co-repressor. Depending on the situation, the co-binding molecule may bind to a complex of the binding molecule and a low-molecular-weight compound, only the binding molecule, or only the low-molecular-weight compound.

Binding molecules thus recovered may be analyzed and identified by analytical methods such as mass spectrometry, amino acid analysis, etc. (cf., for example, U.S. Pat. No. 5,955,729). In the case of the phage library, the recovered phage can be amplified to analyze the inserted gene. Using the present method, it is possible to identify unknown binding molecules having the activity to bind to an immobilized low-molecular-weight compound. Novel binding molecules identified by this method are useful as a targeting molecule for drugs.

Newly identified binding molecules can be used for screening compounds having similar activity as that of an immobilized low-molecular-weight compound used for identification of the binding molecules. That is, binding of the newly identified binding molecule to test substances or the competitive reaction with the low-molecular-weight compound used for identification may be measured. These screenings can be carried out by any methods including the SPR measuring method.

There is no particular limitation on test substances and test samples used for screening, including, for example, peptides, purified or crude proteins, non-peptidic compounds, synthetic compounds, microbial fermentation products, marine creature extracts, plant extracts, cell extracts, etc. These test substances may be either novel or known compounds.

Novel low-molecular-weight compounds thus discovered are expected to have similar physiological activity as the known low-molecular-weight compound and are thus useful as drugs. Compounds obtained using the screening methods of this invention can be used as drugs for humans and other mammals including, for example, mice, rats, guinea pigs, rabbits, chickens, cats, dogs, sheep, pigs, cattle, monkeys, baboons, and chimpanzees, and can be administered according to the commonly used means. Pharmaceutical compositions containing compounds obtained by the screening methods of this invention as an effective ingredient may be prepared by combining the compounds with pharmaceutically acceptable excipients, stabilizers, etc. according to the methods known to those skilled in the art.

The present invention relates to the use of chips on which a low-molecular-weight compound is immobilized for the measurement of its volume changes due to the binding between the low-molecular-weight compound and a binding molecule. This invention has enabled the highly reliable measurement of volume changes by chips on which low-molecular-weight compounds are immobilized. In detection methods using volume changes as an indicator, the reliability of analysis is enhanced by binding a molecule having the molecular weight as high as possible. The present methods have enhanced the reliability of assay results by binding a relatively high molecular weight binding molecule to an immobilized low-molecular-weight compound used as a sensor, thereby enabling detecting the binding reaction between a low-molecular-weight compound and a binding molecule in terms of volume changes, which has been impossible by conventional known methods. The methods of the present invention measure a binding molecule which binds to a low-molecular-weight compound based on volume changes. Furthermore, this invention makes it possible to search for low-molecular-weight compounds binding to targeting molecules, or binding molecules binding to low-molecular-weight compounds using volume changes as an indicator. The use of volume changes as an indictor makes it possible to utilize SPR which enables a speedy measurement with a small amount of samples.

In this invention, multiple low-molecular-weight compounds can be immobilized on the same carrier. Sensor chips serve as the carrier for SPR measurement. Two or more kinds of low-molecular-weight compounds may be mixed and immobilized on the surface of sensor chips, or separately immobilized in the predetermined different positions. A mixture of multiple low-molecular-weight compounds can be evenly immobilized on sensor chips. In this case, binding activities of test compounds to multiple low-molecular-weight compounds can be measured by comparing intensities of SPR signals.

For example, when two kinds of low-molecular-weight compounds different in structure are mixed, immobilized, and then reacted with a test substance, SPR intensity of a test substance binding to only one of the two kinds of immobilized low-molecular-weight compounds is reduced to about a half as compared with that of the other test substance binding to both of them. When low-molecular-weight compounds different in structure are mixed and immobilized, the number of kinds of low-molecular-weight compounds are preferably 20 or less, more preferably 10 or less, further more preferably 5 or less, and most preferably 3 or less. The sensor chip surface may be arbitrarily compartmentalized, and immobilization positions of each low-molecular-weight compounds thereon may be specified beforehand. In this case, only one kind of low-molecular-weight compound is immobilized in the specified position. Therefore, each of low-molecular-weight compounds is immobilized in the different specified positions on the sensor chip, and changes in SPR signals occurring at the specified position may be examined.

Number of molecular species of low-molecular-weight compounds to be immobilized on the sensor chip surface is preferably 4 or more, more preferably 10 or more, further preferably 100 or more, and most preferably 1000 or more per 1 cm². Multiple molecules can be immobilized at specified positions by techniques for preparing DNA chips (Schena, M. et al. (1995) Science, vol. 270, p 467; Shalon, D. et al. (1996) Genome Res., vol. 6, p 639; Lemieux, B. et al. (1998) Mol. Breeding, vol. 4, p 277; Fodor, S. P. A. et al. (1991) Science, vol. 251, p 767; Blachard, A. (1998) In Genetic Engineering, principles and methods (ed. J. Setlow), vol. 20, Plenum Press; Khrapko, K R. et al. (1991) Mol. Biol., vol. 25, 581; Schena, M. et al. (1998) Trends Biotechnol., vol. 16, p 301).

Multiple low-molecular-weight compounds structurally analogous to, for example, glucocorticoids, mineralocorticoids, estrogens, androgens, vitamin D3 and its derivatives, etc. may be immobilized at specified positions on the same sensor chips, and simultaneously reacted with a test substance to measure changes in SPR of immobilized compounds at each position. Thus, it becomes possible to detect the specificity of a test substance for each of low-molecular-weight compounds. When a mixture of receptors for each of low-molecular-weight ligands and a test substance are simultaneously reacted with the above-described chips, specific agonists or antagonists can be identified. The present methods are capable of simply measuring a lot of test substances in a short time, and useful as screening methods.

As a specific embodiment of the present invention, a vitamin D3 derivative can be used as a low-molecular-weight compound and a vitamin D3 receptor can be used as a binding molecule or a targeting molecule.

Vitamin D derivatives have various biological activities such as an osteogenic effect, and have been developed as pharmaceuticals for bone related diseases. Vitamin D derivatives are known to exhibit various biological activities by binding to vitamin D receptors that exist in a nucleus. Therefore, to develop novel vitamin D3 derivatives, it is important to determine binding activities of the vitamin D3 derivatives to vitamin D3 receptors. Though several methods for determining the binding activities of vitamin D3 derivatives to vitamin D receptors, such as those using radioisotopes, no method is available for convenient and high sensitive detection of a large amount of test compounds. For example, a binding assay between vitamin D receptors s and ³H-labeled vitamin D is widely used, but this assay system has some problems, such as use of a radioisotope, complicated operation, a difficulty of detecting a subtle difference in activity between prepared protein lots.

Furthermore, an SPR assay could not be applied to detection of interaction between vitamin D3 derivatives and compounds that bind to vitamin D3, because immobilization of vitamin D3 derivatives on a chip was difficult.

The present inventor found that vitamin D3 derivatives can be immobilized on a sensor chip via a linker and the binding activity between vitamin D3 derivatives and vitamin D3 receptors can be assayed using the methods of this invention.

Herein, vitamin D3 derivatives refer to compounds that have the 9,10-secocholesta-5,7,10(19)-triene structure. Preferably, they have the (5Z,7E)-9,10-secocholesta-5,7,10(19)-triene structure. More preferably, they have the (1a,5Z,7E)-9,10-secocholesta-5,7,10(19)-trien-1-ol structure, yet more preferably the (1a,5Z,7E)-9,10-secocholesta-5,7,10(19)-trien-1,25-diol structure, yet still more preferably the (1a,3b,5Z,7E)-9,10-secocholesta-5,7,10(19)-trien-1,3-diol structure, and most preferably the (1a,3b,5Z,7E)-9,10-secocholesta-5,7,10(19)-trien-1,3,25-triol structure. Vitamin D3 derivatives of the present invention are not particularly limited, as long as they have the structure described above. The derivatives include the above compounds having various substituents. Vitamin D3 derivatives of the present invention also include functional analogues and structural analogues of the above compounds. Herein, the term “fucntional analogue” refers to a compound that has similar biological activities to those of the above. Compounds that have similar biological activities to those of the above compounds are included in the functional analogues, regardless of the intensity of the activities. The term “structural analogue” used herein refers to a compound that corresponds to the above compounds with various modifications on their structure. Structural analogues can be synthesized artificially, or may be naturally occurring compounds.

A linker that is used to bond a vitamin D3 derivative to a carrier is a moiety that forms a covalent bond with either or both of them, or a compound that is required to form the moiety. The linker may have any chemical structure such as a hydrocarbon chain, a peptide chain, or a sugar chain. In the case of a hydrocarbon chain, it may be a substituted or unsubstituted alkyl group. The linker may have one or more oxygen atoms, nitrogen atoms, or sulfur atoms in the hydrocarbon chain. For example, it may have multiple repeating structures of ethyl or propyl groups via oxygen atoms. Substituents are not particularly limited, and include alkyl, halogen, hydroxy, amino, and carboxyl. It is preferred that the ends of a linker has a suitable substituent, so that it can form a covalent bond with a vitamin D3 derivative and/or a carrier. The suitable substituent includes amino, hydroxy, and thiol. The substituent to be used may be selected according to functional groups on a vitamin D3 derivative and/or a carrier to which it is bound, but preferably the substituent is amino or thiol.

As described above, a vitamin D3 derivative can be immobilized on a carrier such as an SPR sensor chip directly by the covalent bond or indirectly. For indirect immobilization, a combination of two kinds of molecules that are known to bind to each other may be used as the linkers. Such a combination of molecules includes biotin-avidin, and antigen-antibody. The selection and manufacture of the suitable linkers are known to those skilled in the art.

The length of the linker is, in terms of the carbon chain, preferably 50 atoms or less, more preferably 30 atoms or less, still more preferably 20 atoms or less, and most preferably 10 atoms or less. For readily contacting a vitamin D3 derivative with a vitamin D3 receptor, the length of the linker is, in terms of the carbon atoms, preferably not less than 3 atoms.

Any site of a vitamin D3 derivative may form a covalent bond with the linker, as long as a vitamin D3 derivative bound to the linker can bind to a vitamin D3 receptor. A preferable linker-binding site is a position other than position 1 and/or 3. More preferably, it is a carbon atom at position 2, 20, 21, 22, 23, 24, 25, or 26 on the vitamin D3 skeleton, and most preferably, it is a carbon atom at position 2, 25, or 26.

Herein, vitamin D3 receptors are defmed as molecules having the binding activities to vitamin D3 derivatives. Vitamin D3 receptors used herein include not only known vitamin D3 receptors that bind to vitamin D3 derivatives but also so called vitamin D3 receptor-like compounds that have similar structures or activities to the known vitamin D3 receptors. Usually, vitamin D3 receptors are proteins that exist naturally in vivo and have binding activities to naturally occurring ligands, but are not limited thereto. Vitamin D3 receptors used herein also include the above-described receptor proteins that have been artificially or spontaneously modified as long as they bind to naturally occurring ligands. The receptors may be those existing in blood or cells. Intracellular receptors include membrane receptors, nuclear receptors, and cytoplasmic receptors. The vitamin D3 receptors used in the methods of the present invention may be proteins, non-peptide compounds, low-molecular-weight compounds, organic compounds, etc. Preferably, they have a molecular weight necessary for the detection of changes in SPR signals. Such a molecular weight is defined as described above.

Interaction between vitamin D3 derivatives and vitamin D3 receptors can be measured by the methods as described above. In one specific embodiment of the methods of this invention, the binding of a vitamin D3 derivative to a vitamin D3 receptor can be detected by contacting a vitamin D3 derivative immobilized on a carrier via a linker with a vitamin D3 receptor and measuring a volume change resulting from the formation of the complex between the immobilized vitamin D3 derivative and a vitamin D3 receptor.

In another specific embodiment, the content of a vitamin D3 receptor in a sample can be measured by contacting a vitamin D3 derivative immobilized on a carrier via a linker with a sample and measuring a volume change resulting from the binding of the vitamin D3 derivative to the vitamin D3 receptor.

Not only vitamin D3 receptors but also vitamin D3 derivatives contained in a sample can be measured. This measurement can be performed by contacting a predetermined amount of a vitamin D3 receptor with a vitamin D3 derivative immobilized on a carrier via a linker, in the presence of or after the contact with a sample, and measuring a volume change resulting from the binding of the vitamin D3 derivative to the vitamin D3 receptor. The phrase “a predetermined amount” used herein does not specifically have an upper limit or a lower limit, but is usually in a range of from 20 ng to 3000 ng.

In still another embodiment, the binding activity of a test compound to a vitamin D3 receptor can be detected by contacting a vitamin D3 derivative immobilized on a carrier via a linker with a vitamin D3 receptor in the presence of a test compound, and measuring a volume change resulting from the binding of the immobilized vitamin D3 derivative to the vitamin D3 receptor.

Furthermore, compounds that bind to vitamin D3 receptors that are targeting molecules can be screened using the above-mentioned methods for detecting the binding activity of a test compound to a vitamin D3 receptor. The methods determine the binding activity of a test compound to a vitamin D3 receptor and select a compound having the binding activity to the vitamin D3 receptor.

Compounds that are screened by the methods as described above are also within the scope of the present invention. Such compounds are useful as agonists, antagonists, inhibitors, or accelerators for vitamin D3 receptors, or as ligands that allow detection or purification of vitamin D3 receptors. Vitamin D3 receptor agonists or antagonists are useful as pharmaceutical agents. Particularly, these compounds would be useful as pharmaceutical agents for treating bone related diseases such as osteoporosis and bone metastasis of malignant tumor.

Furthermore, the present invention provides a carrier on which one or multiple kinds of vitamin D3 derivatives have been immobilized. The carriers include a carrier for measuring SPR such as an SPR sensor chip, on which one or multiple kinds of vitamin D3 derivatives have been immobilized via a linker. Such chips are useful because they enable a reliable measurement of volume changes. Compounds that bind to vitamin D3 receptors or novel vitamin D3 receptors that bind to vitamin D3 derivatives can be screened using volume changes as an index. SPR is available for rapidly measuring volume changes using a small amount of samples.

All patents and publications cited herein are incorporated by reference.

The present invention is illustrated in detail below with reference to example, but is not to be construed as being limited thereto.

### EXAMPLE 1

1. Apparatus and Reagents

BIACORE up grade, BIACORE 3000, Sensor Chip CM5, HBS (containing 10 mM Hepes, 0.15 M NaCl, 3.4 mM EDTA, and 0.05% Tween 20, pH 7.4), and amine coupling kit were purchased from Biacore AB.

2. Synthesis of Low-Molecular-Weight Compound to be Immobilized

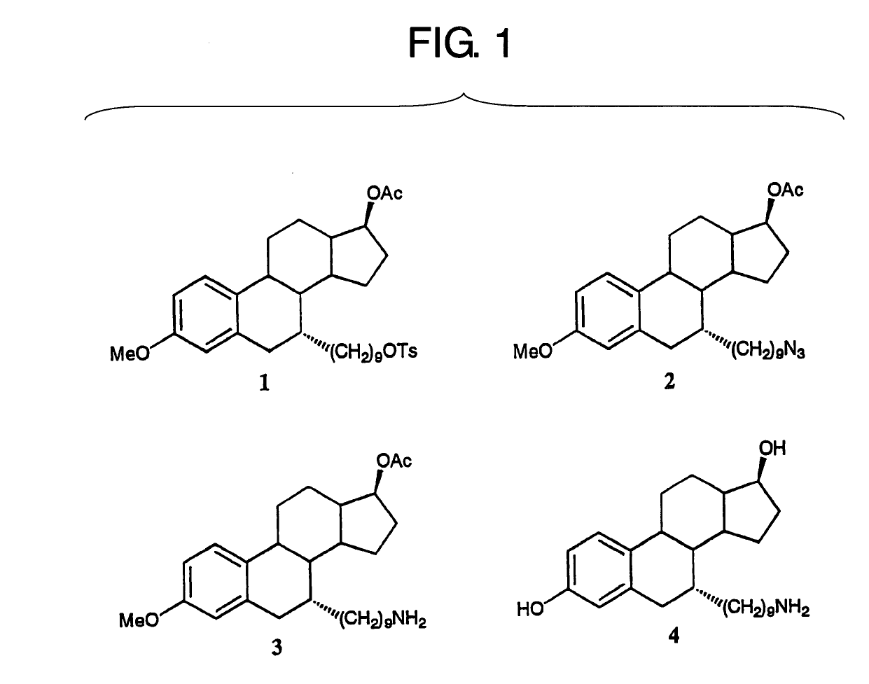

Compound 4 [7α-(9-aminononyl)estradiol] to be immobilized on the Sensor Chip CM5 was synthesized as follows. Compound 1 (34 mg) was synthesized according to the method described in a patent (U.S. Pat. No. 4,659,516). A NaN₃ (3.9 mg) was added to a solution of compound 1 in DMF (1 ml), and the mixture was stirred at 50° C. for 1.5 hr. After the solvent was evaporated, the residue was dissolved in CH₂Cl₂, washed with water, and dried over MgSO₄, and then the solvent was evaporated. The resulting solid was purified by preparative TLC to obtain compound 2 (22 mg, yield 82%). Compound 2 (20 mg) was dissolved in MeOH (2 ml), and stirred under a hydrogen atmosphere in the presence of 10% Pd—C catalyst for 2 hr. The reaction mixture was filtered through a celite, and the filtrate was concentrated to obtain compound 3 (15 mg, yield 79%) as solid. A solution of DIBAL-H in toluene (1 M, 4 ml) was added dropwise to a solution of compound 3 (48 mg) in toluene (3 ml) under ice-cooling, and the resulting mixture was stirred for 5 min followed by heating at reflux for 5 hr. A Roche salt solution (2 ml) and tetrahydrofuran (5 ml) were added to the resulting solution, and the mixture was stirred at room temperature overnight. The organic layer was dried over MgSO₄, and then concentrated in vacuo to obtain compound 4 [7α-(9-aminononyl)-estradiol; 7α-(9-aminononyl)-3,17-α-dihydroxy-estra-1,3,5(10)-triene-(7α-(9-aminononyl)estradiol)]] (41 mg, yield 99%) as solid. ¹H NMR δ: 0.70-3.90 (m, 37H), 5.2-5.6 (m, 1H), 6.4-7.4 (m, 3H). Structures of compounds 1, 2, 3, and 4 are shown in FIG. 1.

3. Preparation of Estrogen Receptor

The fusion protein of a partial sequence containing the ligand binding domain of human estrogen receptor-α, ERLBD, (Ala288-Arg555) (Krust, A., Green, S., Argos, P., Kumar, V., Walter, P., Bornert, J. M., and Chambon, P. (1986) EMBO J., vol. 5, pp. 891-897) with the maltose binding protein (MBP-ERLBD) were prepared as follows.

The cDNA encoding ERLBD (Ala288-Arg555) was introduced into the pMAL-c2 plasmid (New England BioLabs), and the expression vector thus obtained was transferred into the Escherichia coli strain JM109. The transformants were selected on agar plates containing ampicillin. The transformant strain was cultured in LB medium (750 ml) containing ampicillin (500 μg/ml) at 37° C. and the expression of MBP-ERLBD was induced by adding IPTG (final concentration 0.3 mM). After the expression was induced, the strain was continuously cultured overnight, and then centrifuged (3000 rpm×15 min) to collect cells. The cells thus obtained were suspended in 60 ml of buffer A (20 mM Tris-HCl containing 1 mM EDTA and 1 mM dithiothreitol, pH 7.5), sonicated (Duty cycle 50%, 30 sec, 10 times) on ice, and centrifuged (12,000 rpm×20 min) to recover MBP-ERLBD in the supernatant. The supernatant was loaded onto a Q-Sepharose fast flow column (50 ml, Pharmacia) equilibrated with buffer A, and MBP-ERLBD bound to the column was eluted with a linear gradient of NaCl concentration in buffer A. Fractions containing MBP-ERLBD were collected, loaded to an Amylose column (30 ml, New England BioLabs) equilibrated with buffer A containing 0.15 M NaCl. After washing the column with the same buffer, MBP-ERLBD was eluted with buffer A containing 10 mM maltose and 0.15 M NaCl (purified MBP-ERLBD).

Factor Xa S/E=100 was added to MBP-ERLBD thus obtained, and the mixture was stood at 4° C. overnight to cleave the bond between MBP and ERLBD. To remove Factor Xa and maltose, the reaction mixture was loaded onto a Q-Sepharose fast flow column (20 ml) equilibrated with buffer A. After washing the column with the same buffer, ERLBD and MBP were eluted with buffer A containing 0.2 M NaCl. Eluted fractions were loaded onto an Amylose column (30 ml) equilibrated with buffer A containing 0.15 M NaCl to adsorb MBP to the column, and ERLBD was obtained as the unbound fraction (purified ERLBD).

The purified preparations of MBP-ERLBD and ERLBD described above as well as the full-length human estrogen receptor-α (Pan Vera Corp.) were used for the following experiments.

4. Immobilization of 7α-(9-aminononyl)Estradiol on the Sensor Chip

Immobilization of 7α-(9-aminononyl)estradiol on the Sensor Chip CM5 (Pharmacia) was carried out as follows. HBS was used as a continuous flow buffer, and the flow rate was set at 5 μl/min. Carboxyl groups of carboxymethyl dextran on the Sensor Chip CM5 were activated by injecting 100 μl of 0.05 M N-hydroxysuccinimide (NHS)/0.2 M N-ethyl-N′-(3-dimethyl aminopropyl)-carbodiimide (EDC). 7α-(9-aminononyl)estradiol (approx. 7 mg/ml in benzene, approx. 4.5 mg/ml in 2-PrOH) was first diluted 10-fold in EtOH, further diluted 10-fold using 10 mM Na-acetate buffer, pH 4.0 or pH 4.5. Aliquots (10 to 50 μl) were injected onto the chip and immobilized by the amine coupling method. Then, excessive activated groups were blocked by injecting 100 μl of ethanolamine, pH 8.5. Further, non-covalently bound substances were washed out by 10 μl of 0.1 M Gly-HCl buffer, pH 2.5 and 10 mM HCl.

5. Assay of Binding Activities of MBP-ERLBD and ERLBD to the Immobilized 7α-(9-aminononyl)estradiol

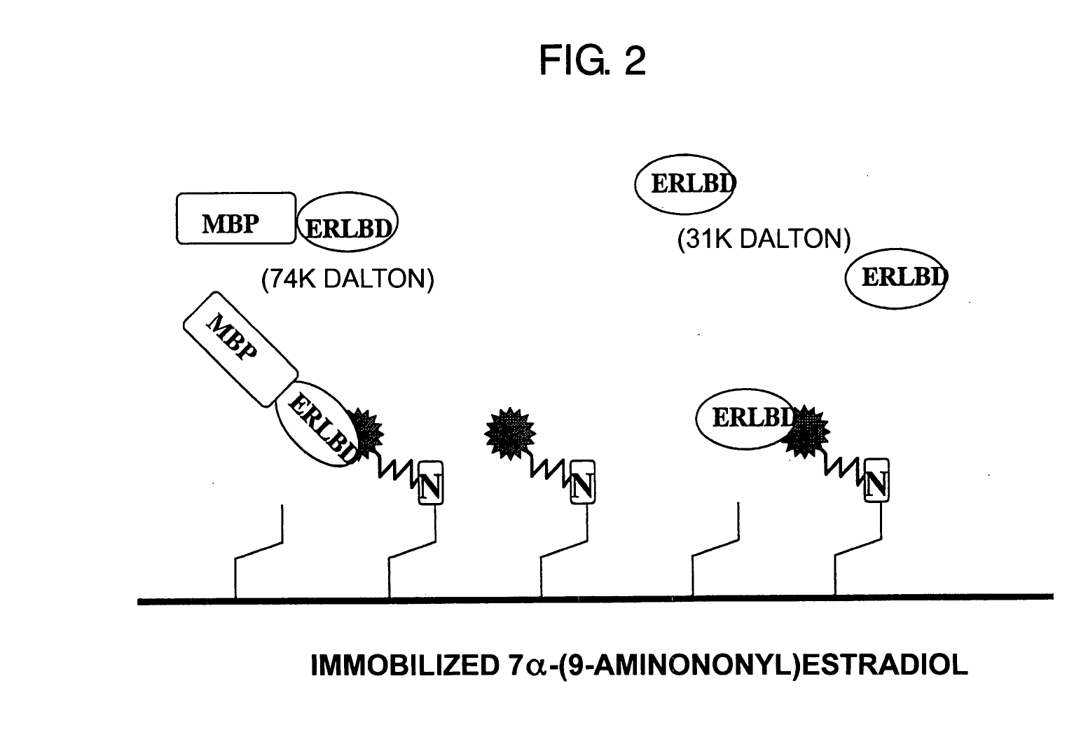

The reaction principle was illustrated in FIG. 2. MBP-ERLBD (20 μg/ml, 10 μl) or ERLBD (20 μg/ml, 10 μl) which were prepared by the above-described method were injected onto the chip to assay their binding activities to the immobilized 7α-(9-aminononyl)estradiol. Regeneration and washing of sensor chips were performed by injecting 10 μl of 7% propanol/50 mM HCl.

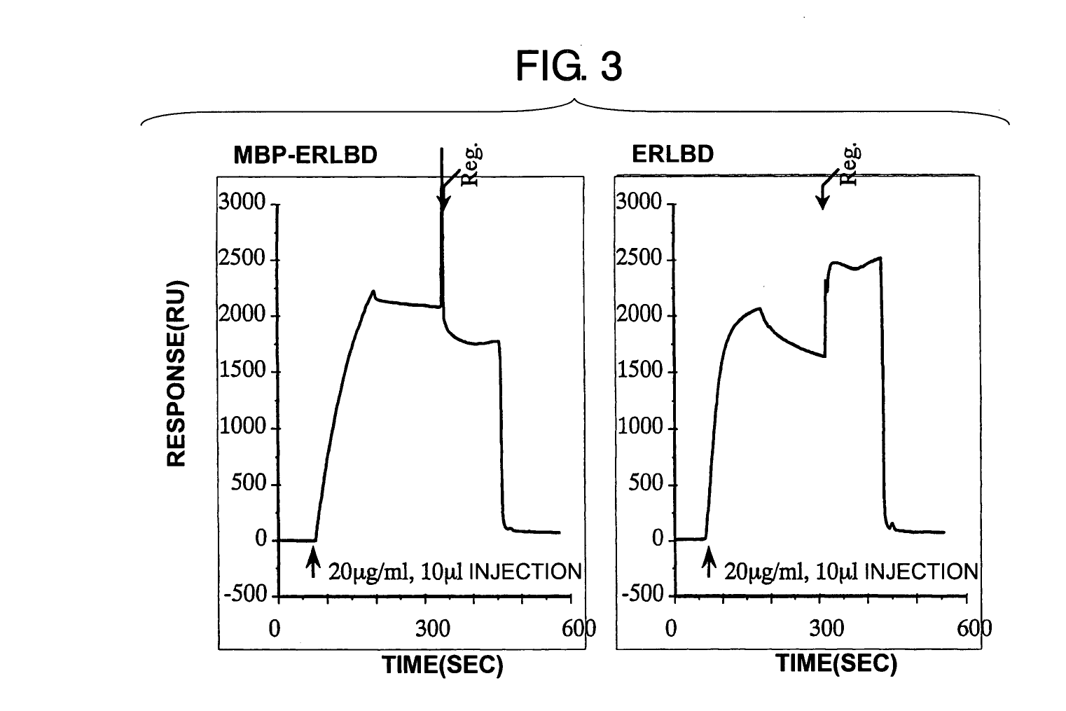

As a result, the binding level of about 2000 RU was detected for both MBP-ERLBD and ERLBD, indicating that binding activities of MBP-ERLBD and ERLBD are almost equal (FIG. 3).

For the purpose of measuring the reproducibility and lot-to-lot variations of the binding activity assay, the binding activity assay was performed using different lots of MBP-ERLBD and ERLBD preparations to compare assay values in duplicate. The binding levels of MBP-ERLBD (Lot #90) (20 μg/ml, 10 μl) were 2124.9 and 2048.9 RU, indicating a high reproducibility (% CV=2.6%) (Table 1). Highly reproducible data were also obtained for ERLBD (Lots. #110, #111, #112, and #113) (20 μg/ml, 10 μl each), as shown in Table 1. These results indicate that, as compared with the case of immobilization of proteins, sensor chips on which low-molecular-weight compounds are immobilized can be regenerated for re-use without causing any changes in the activity and structure of the immobilized compounds even after washing. The results also indicate that binding activities can be accurately compared lot-to-lot variations, and that this method may be applied to the product quality control, etc. This method has also enabled easily monitoring active fractions in the purification process of ERLBD, leading to shortening of its preparation time. Results of examination of the reproducibility of the binding activity assay and the comparison thereof among sample lots are summarized in Table 1.

TABLE 1
Reproducibility of Binding Activity - BIACORE method -
analyte: 20 μg/ml, 10 μl, Injection

| | Response [RU] | (n = 2) | % CV |
|---|---|---|---|
| MBP-ERLBD | | | |
| #90 | 2124.9 | 2048.9 | 2.6 |
| ERLBD | | | |
| #110 | 1825.8 | 1815.0 | 0.4 |
| #111 | 1032.0 | 990.3 | 2.9 |
| #112 | 1255.7 | 1203.9 | 3.0 |
| #113 | 1613.7 | 1597.3 | 0.7 |

Using MBP-ERLBD (Lot #90) and ERLBD (Lots. #110, #111, #112, and #113) as the binding molecule, the binding activity was assayed in duplicate. RU is the unit of surface plasmon resonance intensity. % CV represents the dispersion in duplicate, indicating the larger the value, the greater the dispersion.

6. Confirmation of Specific Interaction

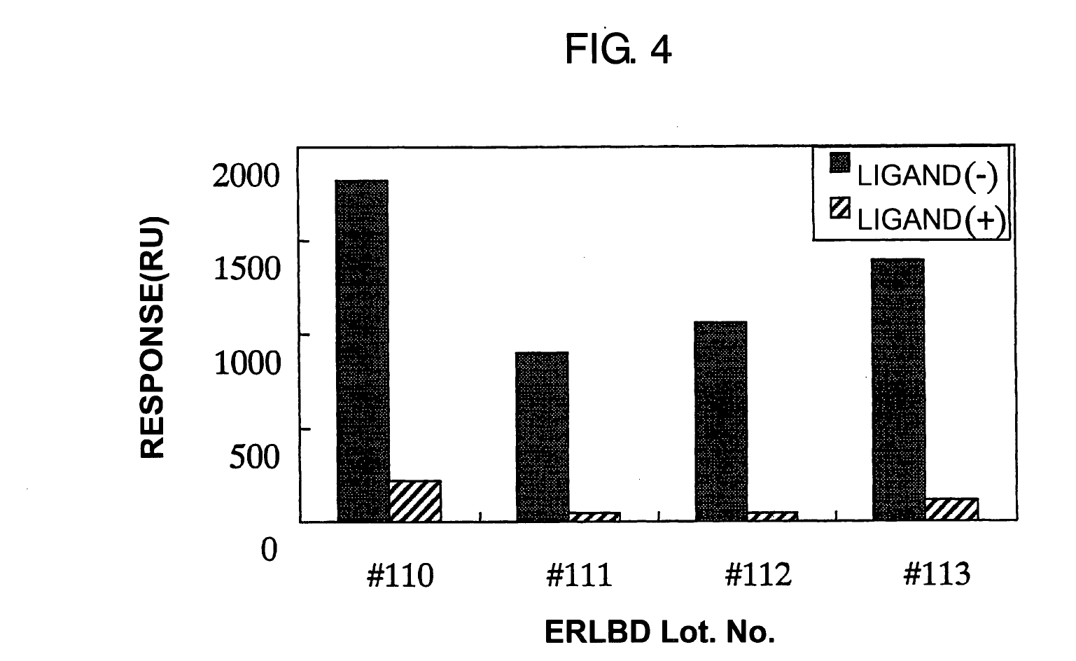

A sample was prepared by reacting 20 μg/ml (0.645 μM) of ERLBD with 363 μM of β-estradiol (SIGMA), which is about 500-fold molar concentration of ERLBD, for 1 hr at room temperature. Another sample was prepared in the same manner without using β-estradiol. The binding activities of these samples to the immobilized 7α-(9-aminononyl)estradiol was examined and compared. As shown in FIG. 4, the addition of excess β-estradiol remarkably reduced the binding level of ERLBD to the immobilized 7α-(9-aminononyl)estradiol. These results indicate that the binding of ERLBD used as the binding molecule to the immobilized 7α-(9-aminononyl)estradiol is competitively inhibited by the specific binding of β-estradiol used as a ligand. The results also indicate that, in the binding activity assay performed using a mixture of the binding molecule ERLBD and its competitor, the binding molecule retains the appropriate binding activity. The results further indicate that, even when only the ligand binding domain is used together with the binding molecule, the binding activity can be meaningfully assayed.

7. Binding Activity Assay by the Conventional Method Using Labeled Oestradiol

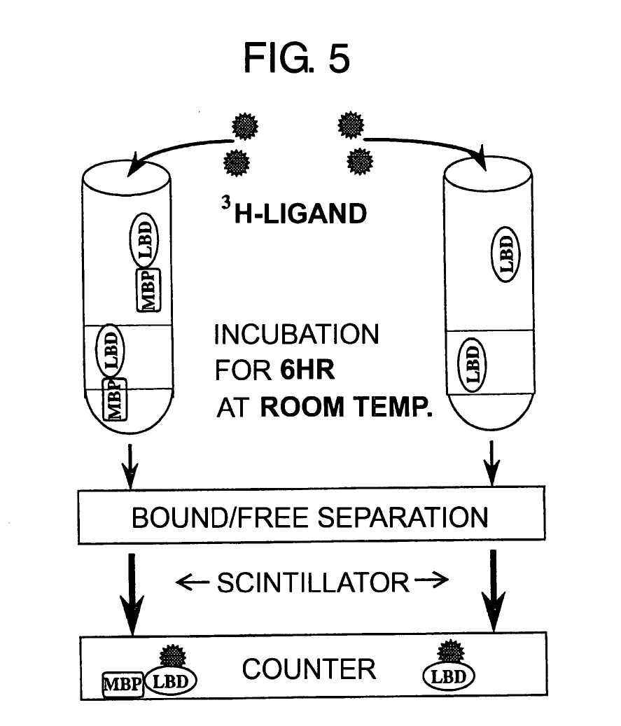

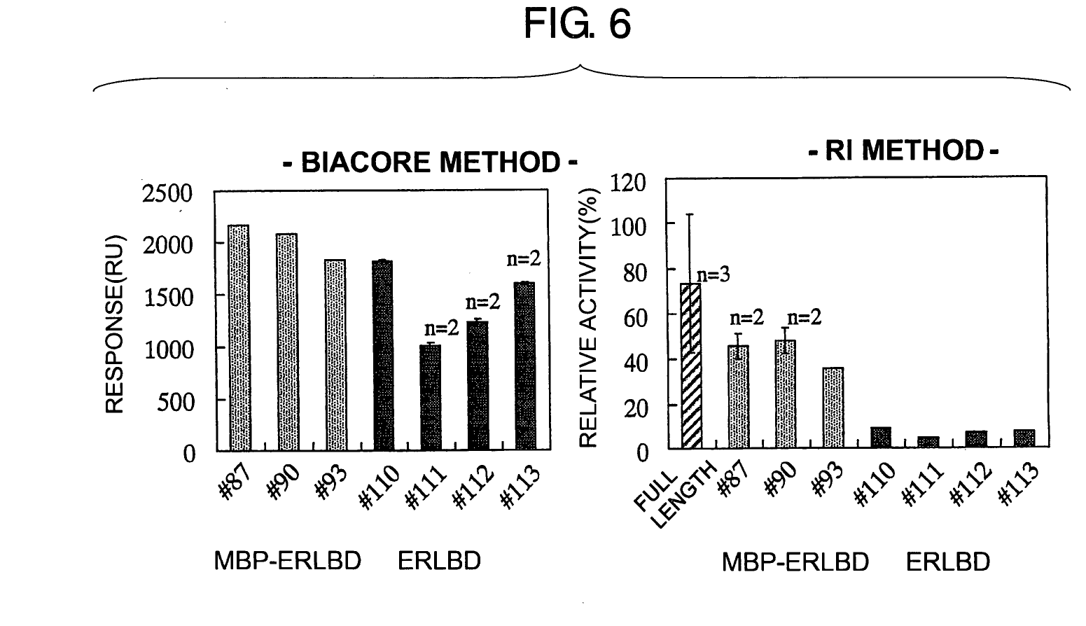

The binding activity of [6,7-³H]oestradiol (Amersham Pharmacia Biotech) to a full-length human estrogen receptor-α, ERLBD, and MBP-ERLBD was assayed according to a conventional method using a ligand labeled with radioisotope (RI) (Sasson, S. and Notides, A. C. (1988) Mol. Endcrinol., Vol. 2, No. 4, p307-312). The reaction is schematically illustrated in FIG. 5. The RI value obtained when [6,7-³H]oestradiol bound to all the ERLBD added to the assay system was taken as 100%, and the relative binding activity was calculated. Reproducibility of the binding activity detected by the conventional RI method is summarized in Table 2. A full-length human estrogen receptor-α and MBP-ERLBD (lot #87 and #90) were used as the binding molecule. As shown in Table 2, the binding activities of a full-length human estrogen receptor-α and MBP-ERLBD to the labeled oestradiol greatly differed in every assay, proving that reproducibility of binding activity by this RI method is lower as compared with the method of the present invention. Also, as shown in FIG. 6, in the conventional RI method, only the binding activity of ERLBD was as low as about 10% of the expected value. Since it takes 6 hr for the assay by the conventional RI method, conformational changes may occur in proteins during the assay, resulting in a decrease in the binding activity. The method of the present invention uses proteins as the binding molecule, and the reaction time is thus short, so that the binding activity can be measured with a good reproducibility without causing a decrease in the binding activity.

TABLE 2
Reproducibility of Binding Activity - RI method -

| | Relative activity (%) | | | |
|---|---|---|---|---|
| Full length receptor | (n = 3) | | | % CV |
| | 39.8 | 98.9 | 81.6 | 41.4 |

| | Relative | | |
|---|---|---|---|
| | activity | | |
| MBP-ERLBD | (%) (n = 2) | | % CV |
| #87 | 42.9 | 49.9 | 12.2 |
| #90 | 41.1 | 52.0 | 11.6 |

### EXAMPLE 2

1. Apparatus and Reagents

BIACORE 3000, Sensor chip CM5, HBS-EP (10 mM Hepes, 0.15 M NaCl, 3.4 mM EDTA, 0.05% Tween 20, pH 7.4), and amine coupling kit were purchased from Biacore AB. Recombinant human Vitamin D3 receptor (VDR) was purchased from Pan Vera Corp.

2. Synthesis of an aminoalkyl vitamin D3 derivative, ED-533

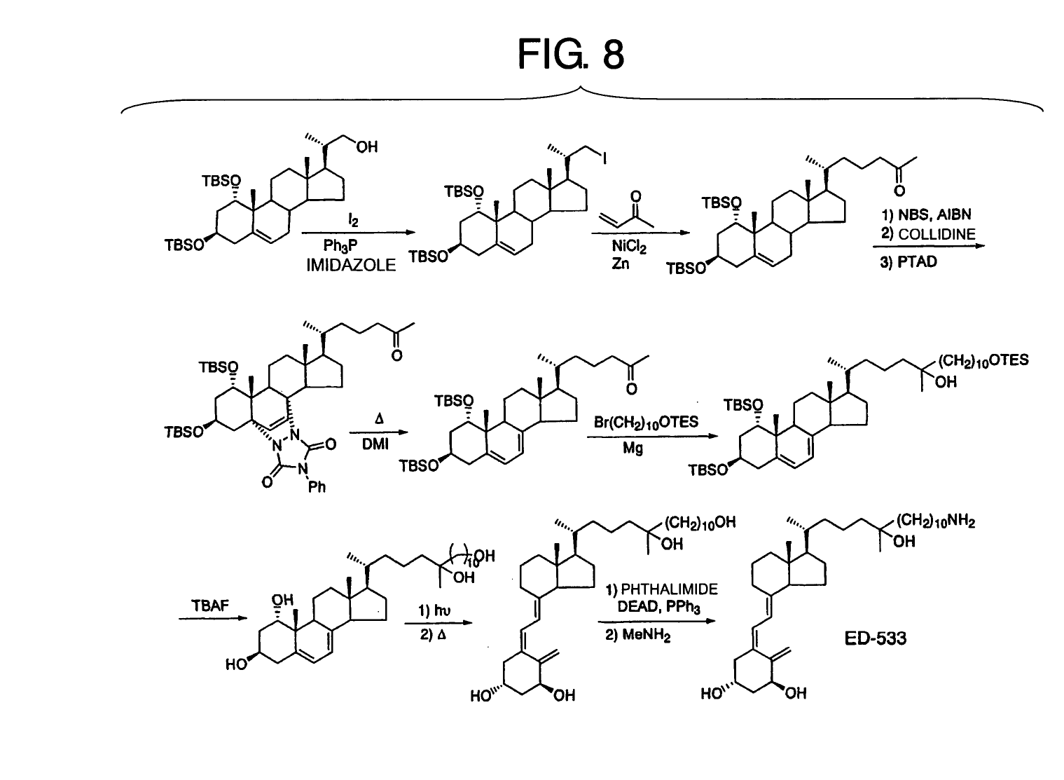

ED-533 ((1α,3β,5Z,7E)-25-(10-aminodecanyl)-27-nor-9,10-secocholesta-5,7,10(19)-triene-1,3,25-triol) was prepared as follows. The synthetic scheme is shown in FIG. 8.

(1) Preparation of (1α,3β,20S)-1,3-bis((1,1-dimethylethyl)dimethylsilyl)oxy)-20-iodomethyl-pregna-5-ene

To a mixture of (1α,3β,20S)-1,3-bis((1,1-dimethylethyl)dimethylsilyl)oxy)-pregna-5-ene-20-methanol (Chem. Pharm. Bull. 39(12), 3221 (1991), 34.14 g), triphenylphosphine (18.62 g), imidazole (5.24 g), and dichloromethane (350 ml), iodine (16.52 g) was added while being cooled on ice, and the mixture was stirred at room temperature for 30 minutes. The reaction mixture was evaporated under reduced pressure to remove the solvent, hexane was added to the resultant residue, and then the insoluble matter was filtered out. The obtained filtrate was washed with aqueous sodium thiosulfate solution, 0.5 N hydrochloric acid, saturated aqueous sodium bicarbonate solution, and saturated brine, dried over anhydrous sodium sulfate, and then concentrated under reduced pressure. The resultant residue was washed with acetonitrile to yield (1α,3β,20S)-1,3-bis((1,1-dimethylethyl)dimethylsilyl)oxy)-20-iodomethyl-pregna-5-ene (36.63 g, 90%) as a white solid. The NMR data for this compound are as follows:

¹H NMR (CDCl₃) δ: 0.03(3H, s), 0.04(3H, s), 0.05(3H, s), 0.07(3H, s), 0.72(3H, s), 0.88(9H, s), 0.89(9H, s), 0.96(3H, s), 2.13-2.37(2H, m), 3.11-3.21(1H, m), 3.34(1H, d, J=8.9 Hz), 3.77(1H, brs), 3.91-4.06(1H, m), 5.41-5.49(1H, m).

(2) Preparation of 1α,3β-1,3-bis((1,1-dimethylethyl)dimethylsilyl)oxy)-27-nor-5-cholesten-25-one

A mixture of nickel chloride hexahydrate (9 g), zinc powder (12.4 g), methyl vinyl ketone (14.8 g), and pyridine (200 ml) was stirred at 60° C. for 30 minutes, and cooled to room temperature. A mixture of (1α,3β,20S)-1,3-bis((1,1-dimethylethyl)dimethylsilyl)oxy)-20-iodomethyl-pregna-5-ene (20 g), tetrahydrofuran (50 ml), and pyridine (100 ml) was added thereto, and the mixture was stirred at room temperature for 2 hours. The reaction mixture was diluted with ethyl acetate, and filtered through celite. The filtrate was washed with 0.5 N hydrochloric acid, saturated aqueous sodium bicarbonate solution, and saturated brine, dried over anhydrous sodium sulfate, and then concentrated under reduced pressure. The resultant residue was purified by silica gel column chromatography (hexane:ethyl acetate=10:1) to yield 1α,3β-bis((1,1-dimethylethyl)dimethylsilyl)oxy)-27-nor-5-cholesten-25-one (16.8 g, 91%) as a white solid. The NMR data for this compound are as follows:

¹H NMR (CDCl₃) δ: 0.03 (3H, s), 0.04(3H, s), 0.05(3H, s), 0.07(3H, s), 0.67 (3H, s), 0.88(9H, s), 0.88(9H, s), 2.13 (3H, s), 3.77(1H, brs), 3.91-4.06(1H, m), 5.41-5.49(1H, m).

(3) Preparation of a 4-phenyl-1,2,4-triazolin-3,5-dione adduct of 1α,3β-1,3-bis((1,1--dimethylethyl)dimethylsilyl)oxy)-27-nor-5,7-cholestadien-25-one

A mixture of 1α,3β-1,3-bis((1,1-dimethylethyl)dimethylsilyl)oxy)-27-nor-5-cholesten-25-one (16.8 g), N-bromosuccinimide (6.14 g), 2,2′-azobisisobutyronitrile (1.3 g), and hexane (200 ml) was heated to reflux for 15 minutes. The reaction mixture was cooled to room temperature, the insoluble matter was filtered out, and then the solvent was evaporated off under reduced pressure. To the resultant residue, toluene (200 ml) and γ-collidine (13.3 ml) were added at room temperature sequentially, and the mixture was heated to reflux for 2.5 hours. The reaction mixture was cooled to room temperature, and the insoluble matter was filtered out. The mixture was diluted with hexane, washed with 0.5 N hydrochloric acid, saturated aqueous sodium bicarbonate solution, and saturated brine, and then dried over anhydrous sodium sulfate. This mixture was concentrated under reduced pressure. To the resultant residue, dichloromethane (200 ml) and 4-phenyl-1,2,4-triazol-3,5-dione (4.7 g) were added at room temperature sequentially, and the mixture was stirred at room temperature for 45 minutes. The reaction mixture was concentrated under reduced pressure, and the obtained residue was purified by silica gel column chromatography (hexane:ethyl acetate:dichloromethane=10:1:1) to yield a 4-phenyl-1,2,4-triazolin-3,5-dione adduct of 1α,3β-1,3-bis((1,1-dimethylethyl)-dimethylsilyl)-oxy)-27-nor-5,7-cholestadien-25-one (7.8 g, 36%) as a white amorphous. The NMR data for this compound are as follows:

¹H NMR (CDCl₃)δ: 0.07(3H, s), 0.08(3H, s), 0.10(3H, s), 0.13(3H, s), 0.80(3H, s), 0.88(9H, s), 0.89(9H, s), 2.14(3H, s), 3.18-3.30(1H, m), 3.84(1H, brs), 4.69-4.85(1H, m), 6.21(1H, d, J=8.4 Hz), 6.37(1H, d, J=8.4 Hz), 7.34-7.49(m, 5H).

(4) Preparation of 1α,3β-1,3-bis((1,1-dimethylethyl)dimethylsilyl)oxy)-27-nor-5,7-cholestadien -25-one

A mixture of a 4-phenyl-1,2,4-triazolin-3,5-dione adduct of 1α,3β-1,3-bis((1,1-dimethylethyl)dimethylsilyl)oxy)-27-nor-5,7-cholestadien-25-one (5.7 g) and 1,3-dimethyl-2-imidazolidinone (57.1 ml) was stirred at 150°C. under argon atmosphere. The solvent was evaporated off under reduced pressure, and the resultant residue was purified by silica gel column chromatography (hexane:ethyl acetate=8:1) to yield 1α,3β-1,3-bis((1,1-dimethylethyl)dimethylsilyl)oxy)-27-nor-5,7-cholestadien-25-one (3.3 g, 74%) as a light yellow amorphous. The NMR data for this compound are as follows:

¹H NMR (CDCl₃) δ: 0.03-0.08(9H, m), 0.10(3H, s), 0.61(3H, s), 0.88(9H, s), 0.88(9H, s), 2.13(3H, s), 2.69-2.82(1H, m), 3.69(1H, brs), 3.97-4.11(1H, m), 5.25-5.34(1H, m), 5.52-5.61 (1H, m).

(5) Preparation of 1α,3β-1,3-bis((1,1-dimethylethyl)dimethylsilyl)oxy)-25-(10-hydroxydecanyl)-27-nor-5,7-cholestadien-25-ol

To a mixture of magnesium powder (3.3 g), 10-((triethylsilyl)oxy)decanyl bromide (4.8 g), and tetrahydrofuran (30 ml), several drops of ethylene bromide were added at 50° C. under argon atmosphere, and the mixture was stirred for 30 minutes. To the reaction mixture, a mixture of 1α,3β-1,3-bis((1,1-dimethylethyl)dimethylsilyl)oxy)-27-nor-5,7-cholestadien-25-one (2.2 g) and tetrahydrofuran (10 ml) was added at 50° C., and the mixture was stirred at the same temperature overnight. The reaction mixture was cooled to room temperature, saturated aqueous ammonium chloride solution was added thereto, and then the mixture was extracted with ethyl acetate. The organic layer was dried over anhydrous sodium sulfate, and concentrated under reduced pressure. The resultant residue was purified by silica gel column chromatography (hexane:ethyl acetate=10:1) to yield a colorless oil (3.97 g). To a mixture of the resultant colorless oil (3.97 g) and tetrahydrofuran (50 ml), tetrabutylammonium fluoride (1M solution in tetrahydrofuran, 10 ml) was added at room temperature, and the mixture was stirred at the same temperature for 15 minutes. The solvent was evaporated off under reduced pressure, and the resultant residue was purified by silica gel column chromatography (hexane:ethyl acetate=4:1) to yield 1α,3β-1,3-bis((1,1-dimethylethyl)dimethylsilyl)oxy)-25-(10-hydroxydecanyl)-27-nor-5,7-cholestadien-25-ol (1.74 g, 63%) as a white amorphous. The NMR data for this compound are as follows:

¹H NMR (CDCl₃) δ: 0.03-0.08(9H, m), 0.11(3H, s), 0.62(3H, s), 0.88(9H, s), 0.89(9H, s), 2.28-2.41(2H, m), 2.71-2.83(1H, m), 3.57-3.74(3H, m), 3.96-4.13(1H, m), 5.26-5.36(1H, m), 5.54-5.62(m, 1H).

(6) Preparation of (1α,3β,5Z,7E)-25-(10-hydroxydecanyl)-27-nor-9,10-secocholesta-5,7,10(19) -triene-1,3,25-triol

A mixture of 1α,3β-1,3-bis((1,1-dimethylethyl)dimethylsilyl)oxy)-25-(10-hydroxydecanyl)-27-nor-5,7-cholestadien-25-ol (0.5 g) and tetrahydrofuran (350 ml) was irradiated with UV light (transmitted light through 295 nm interference filter, ultraviolet radiation system with 500 W xenon-mercury lamp, Ushio Inc.) for 3 hours, while being stirred with water-cooling under argon stream. Tetrahydrofuran (150 ml) was added to the reaction mixture, and then the mixture was heated to reflux under argon atmosphere for 2 hours. The solvent was evaporated off under reduced pressure, and the resultant residue was purified by silica gel column chromatography (hexane:ethyl acetate=2:1) to yield a mixture (493 mg) comprising (1α,3β,5Z,7E)-1,3-bis((1,1-dimethylethyl)dimethylsilyl)oxy)-25-(10-hydroxydecanyl)-27-nor-9, 10-secocholesta-5,7,10(19)-triene-25-ol. A mixture of the obtained mixture (493 mg), tetrabutylammonium fluoride (1.0 M solution in tetrahydrofuran, 16 ml), and tetrahydrofuran (10 ml) was stirred at 45° C. under argon atmosphere for 2 hours. The reaction mixture was diluted with ethyl acetate, washed with 0.5 N hydrochloric acid, saturated aqueous sodium bicarbonate solution, and saturated brine, dried over anhydrous sodium sulfate, and then concentrated under reduced pressure. The resultant residue was purified by silica gel column chromatography (hexane:ethyl acetate:ethanol=5:5:1) to yield (1α,3β,5Z,7E)-25-(10-hydroxydecanyl)-27-nor-9,10-secocholesta-5,7,10(19)-triene-1,3,25-triol (159 mg, 45%). The physicochemical properties of this compound are as follows:

IR(neat):3338, 2927, 2850, 1711, 1466, 1377, 1261, 1057cm⁻¹; ¹H NMR (CDCl₃) δ: 0.54(3H, s), 2.54-2.65(1H, m), 2.76-2.90(1H, m), 3.63(2H, t, J=6.8 Hz), 4.23(1H, brs), 4.43(1H, brs), 5.00(1H, s), 5.33(1H, s), 6.01(1H, d, J=11.1 Hz), 6.38(1H, d, J=11.1 Hz); MS(EI) m/z 540 (M⁺-H₂O); UV (in EtOH) λₘₐₓ 212.5, 264.5 nm.

(7) Preparation of (1α,3β,5Z,7E)-25-(10-aminodecanyl)-27-nor-9,10-secocholesta-5,7,10(19)-triene-1,3,25-triol

To a mixture of (1α,3β,5Z,7E)-25-(10-hydroxydecanyl)-27-nor-9,10-secocholesta-5,7,10(19)-triene-1,3,25-triol (159 mg), phthalimide (63 mg), triphenylphosphine (89 mg), and tetrahydrofuran (2.8 ml), diethyl azodicarboxylate (0.05 ml) was slowly added at room temperature under argon atmosphere, and the mixture was stirred at room temperature for 1.5 hours. The reaction mixture was concentrated under reduced pressure, and the resultant residue was purified by silica gel column chromatography (hexane:ethyl acetate:ethanol=5:5:1) to yield a crude product (240 mg) containing (1α,3β,5Z,7E)-25-(10-phthalimide decanyl)-27-nor-9,10-secocholesta-5,7,10(19)-triene-1,3,25-triol. A mixture of the resultant crude product (240 mg) and methylamine (40% solution in methanol, 8 ml) was stirred at room temperature under argon atmosphere for one hour. The reaction mixture was purified by thin layer silica gel chromatography (NH TLC plate, Fuji Silysia Chemical, Ltd., dichloromethane:ethanol=10:1) to yield (1α,3β,5Z,7E)-25-(10-aminodecanyl)-27-nor-9,10-secocholesta-5,7,10(19)-triene-1,3,25-triol (ED-533) (8.8 mg, 5.6%) as a white amorphous. The physicochemical properties of this compound are as follows, and the structural formula of ED-533 is represented by formula (1):

IR (neat): 3356, 2927, 2852, 1647, 1466, 1375, 1057cm⁻¹; ¹H NMR (CDCl₃) δ: 0.54(3H, s), 2.66(1H, t, J=7.0 Hz), 2.77-2.88(1H, m), 4.15-4.28(1H, m), 4.35-4.44(1H, s), 4.98(1H, s), 5.32(1H, s), 6.01(1H, d, J=11.1 Hz), 6.36(1H, d, J=11.1 Hz); MS(EI) m/z 539 (M⁺-H₂O); UV (in EtOH) λₘₐₓ 210.8, 263.3 nm. 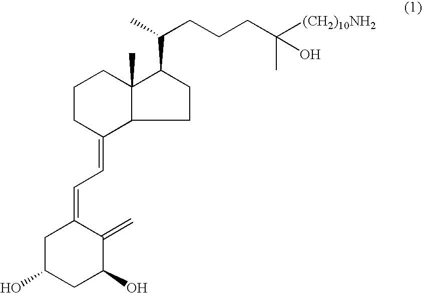
3.Immobilization of ED-533 on the Sensor Chip

ED-533 synthesized in 2 above was immobilized on Sensor Chip CM5 as follows. HBS-EP containing 5% DMSO was used as a running buffer, and the flow rate was set at 5 μl/min. Carboxyl groups of carboxymethyl dextran on the Sensor Chip CM5 were activated by injecting 100 μl of 0.05 M N-hydroxysuccinimide (NHS)/0.2 M N-ethyl-N′-(3-dimethyl aminopropyl)carbodiimide (EDC). ED-533 (1 mg/ml in EtOH) was first diluted 4-fold in EtOH, further diluted 10-fold using 10 mM Na-acetate buffer, pH 4.5. Aliquots (10 to 40 μl) were injected onto the chip and immobilized by the amine coupling method. Excessive activated groups were blocked by injecting 100 μl of ethanolamine, pH 8.5. Non-covalently bound substances were washed out by injecting 10 μl each of 50% aqueous DMSO solution and 50% PrOH/10% DMSO/50 mM HCl.

4. Influences of EtOH and DMSO on Interaction Between Immobilized ED-533 and VDR

VDR was diluted to 100 nM solution with 10% Glycerol/2 mM DTT/HBS-EP. A 10 μL portion of the 100 nM VDR solution containing EtOH or DMSO at the final concentration of 0, 1, 2, or 5%, was injected to the sensor chip. The sensor chip was regenerated and washed with an injection of 5 μL of 15% PrOH/200 mM NaOH.

The results showed that binding activities of VDR were retained when using either 5% EtOH or 5% DMSO. From the results, the following study was conducted using HBS-EP containing 5% DMSO as a running buffer, and using a sample solution containing 5% DMSO.

5. Confirmation of Specific Interaction

To 100 nM VDR, 1,25(OH)₂ vitamin D₃ was added to a fmal concentration of 0, 10, 100, or 1000 nM and the mixture was reacted at room temperature for one hour. A 10 μL portion of the sample solution thus prepared was injected to the sensor chip to examine the binding activity of VDR to immobilized ED-533. After binding signals were obtained, the flow passes except the sensor chip were washed with 50% aqueous DMSO solution, then all the flow passes were washed with an injection of 10 μL of 15% PrOH/5% DMSO/200 mM NaOH.

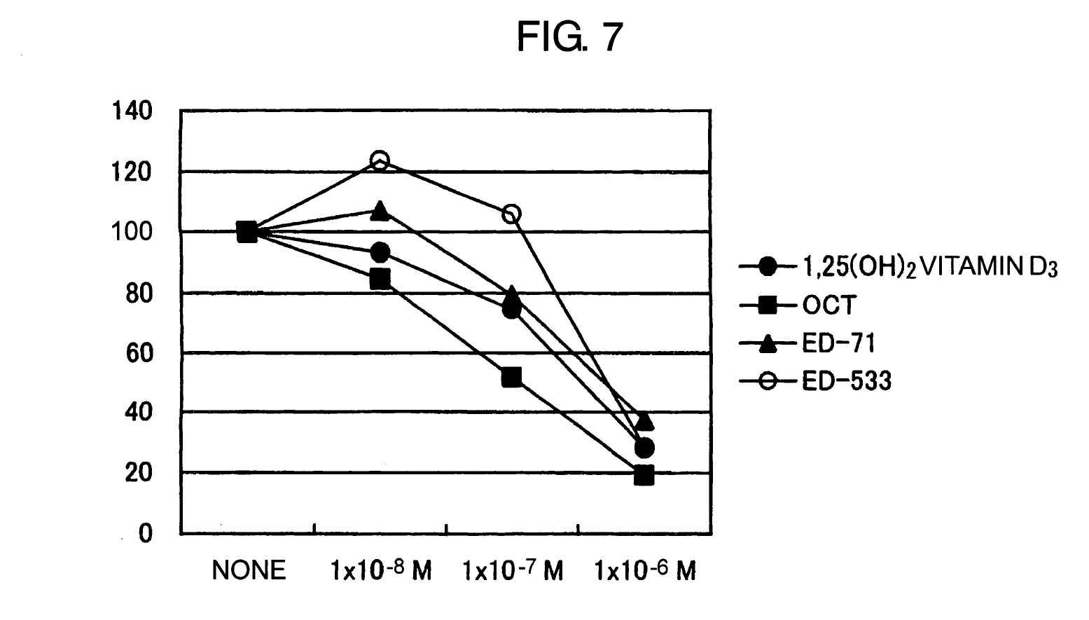

The results showed that 1,25(OH)₂ vitamin D₃ inhibited the interaction of immobilized ED-533 with VDR in a concentration dependent manner (FIG. 7). When 0.1 μM VDR was used, 50% binding inhibition was observeded with the addition of a four-fold higher molar concentration of 1,25(OH)₂ vitamin D₃ than that of VDR. These results indicate that the binding of immobilized ED-533 to VDR is a specific binding via a binding site of VDR to 1,25(OH)₂ vitamin D₃.

6. Determination of Inhibitory Activities of Vitamin D3 Derivatives Against the Binding of Immobilized ED-533 to VDR

VDR (100 nM) and various vitamin D3 derivatives (a final concentration of 0, 10, 100, or 1000 nM) were reacted at room temperature for one hour. A 10 μL portion of the mixed solution was then injected to the sensor chip as a sample to examine the binding activity of the VDR to the immobilized ED-533. After binding signals were obtained, the flow passes except the sensor chip were washed with 50% aqueous DMSO solution, then all the flow passes were washed with an injection of 10 μL of 15% PrOH/5% DMSO/200 mM NaOH. As the test vitamin D3 derivatives, ED-533, OCT (1α,25-dihydroxy-22-oxavitamin D3) (Drugs of the Future 21(12), 1229-1237 (1996)), and ED-71 (2β-(3-Hydroxypropoxy)-1α,25-dihydroxyvitamin D3) (Kittaka A. et al, Organic Letters 2(17), 2619-2622 (2000)) were used. The structural formulae of OCT and ED-71 are represented by formulae (2) and (3), respectively.

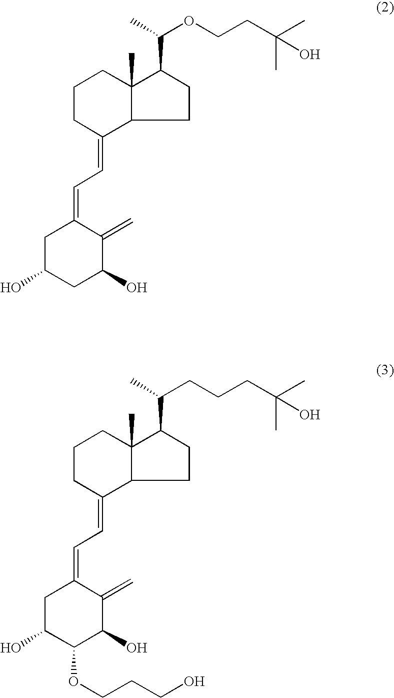

All of ED-533, OCT, and ED-71 had almost the same inhibitory activities to 1,25(OH)₂ vitamin D₃, which is a natural ligand of VDR, indicating that these test compounds had almost the same VDR binding activities (FIG. 7). The IC₅₀ values of these test compounds are shown in Table 3.

TABLE 3

| Compound | IC₅₀ values [μM] |
|---|---|
| 1,25(OH)₂ Vitamin D₃ (First time) | 0.45 |
| 1,25(OH)₂ Vitamin D₃ (Second time) | 0.34 |
| 1,25(OH)₂ Vitamin D₃ (Mean value) | 0.39 |
| OCT | 0.11 |
| ED-71 | 0.50 |
| ED-533 | 0.52 |

1. A method for detecting the binding of a low-molecular-weight compound and a binding molecule which binds to the low-molecular-weight compound, the method comprising:
(1) contacting the immobilized low-molecular-weight compound with the binding molecule to form a complex, and
(2) measuring a volume change due to the binding between the low-molecular-weight compound and the binding molecule.

2. The method according to claim 1, wherein the molecular weight of the low-molecular-weight compound ranges from 50 to 5000.

3. The method according to claim 1, wherein the low-molecular-weight compound is selected from the group consisting of estrogens, androgens, 1,25-hydroxylated vitamin D₃, glucocorticoids, mineralocorticoids, progesterons, thyroid hormones, retinoic acid, and structural and functional analogues thereof.

4. The method according to claim 1, wherein the binding molecule is a protein.

5. The method according to claim 4, wherein the binding molecule is a nuclear receptor.

6. The method according to claim 5, wherein the nuclear receptor is selected from the group consisting of estrogen receptors, androgen receptors, vitamin D₃ receptors, glucocorticoid receptors, mineralocorticoid receptors, progesterone receptors, thyroid hormone receptors, retinoic acid receptors, and orphan receptors.

7. The method according to claim 1, wherein multiple low-molecular-weight compounds are immobilized on the same carrier.

8. The method according to claim 7, wherein the carrier is a sensor chip.

9. The method according to claim 1, wherein the volume change is measured by surface plasmon resonance.

10. The method according to claim 1, wherein the low-molecular-weight compound is immobilized on a sensor chip.

11. A method for measuring a binding molecule contained in a test sample which binds to a low-molecular-weight compound, the method comprising:
(1) contacting the test sample with the immobilized low-molecular-weight compound, and
(2) measuring a volume change due to the binding between the low-molecular-weight compound and the binding molecule.

12. A method for measuring a low-molecular-weight compound which binds to a binding molecule contained in a test sample, the method comprising:
(1) contacting a predetermined amount of the binding molecule with the immobilized low-molecular-weight compound together with a test sample or after contact with a test sample, and
(2) measuring a volume change caused by the binding between the immobilized low-molecular-weight compound and the binding molecule.

13. A method for detecting the binding activity of a low-molecular-weight compound to a binding molecule, the method comprising:
(1) contacting the immobilized low-molecular-weight compound with a binding molecule to form a complex, wherein the low-molecular weight compound is either a test low-molecular-weight compound or a known low-molecular-weight compound capable of binding to the binding molecule, and, when a known low-molecular-weight compound has been immobilized, a complex of a test low-molecular-weight compound with a binding molecule may be formed by any of the following methods (a) to (c):
(a) contacting the known low-molecular-weight compound with the binding molecule in the presence of the test low-molecular-weight compound,
(b) contacting the test low-molecular-weight compound with the binding molecule, and then with the known low-molecular-weight compound, and
(c) contacting the known low-molecular-weight compound with the binding molecule, and then with the test low-molecular-weight compound, and
(2) measuring a volume change caused by the binding between the immobilized known low-molecular-weight compound or immobilized test low-molecular-weight compound and the binding molecule.

14. A method for screening a low-molecular-weight compound capable of binding to a binding molecule, the method comprising detecting the binding activity of the low-molecular-weight compound to the binding molecule by the method according to claim 13 and selecting a compound having the binding activity to the binding molecule.

15. A method for detecting the binding activity of a test low-molecular-weight compound to a binding molecule by the method according to claim 13 using a physiologically or biologically active substance as the binding molecule, wherein the test low-molecular-weight compound functions as agonist, antagonist, inhibitor, or stimulator, of the binding molecule.

16. A method for screening a test low-molecular-weight compound that functions as agonist, antagonist, inhibitor, or stimulator of a binding molecule by the method according to claim 15, the method comprising detecting the binding activity of the test low-molecular-weight compound that functions as agonist, antagonist, inhibitor, and stimulator of the binding molecule, and selecting a compound having the binding activity to the binding molecule.

17. A pharmaceutical composition containing low-molecular-weight compound obtainable by the method according to claim 16.

18. A method for detecting the binding activity of a binding molecule to a low-molecular-weight compound, the method comprising:
(1) contacting the test binding molecule with the immobilized low-molecular-weight compound to form a complex, and
(2) measuring a volume change caused by the binding between the immobilized low-molecular-weight compound and the binding molecule.

19. A method for screening a binding molecule capable of binding to a low-molecular-weight compound, the method comprising detecting the binding activity of the binding molecule to the low-molecular-weight compound by the method according to claim 18, and selecting a compound having the binding activity to the low-molecular-weight compound.

20. The method according to claim 19, wherein said method further comprises recovering and identifying a compound bound to the low-molecular-weight compound.

21. A binding molecule having the binding activity to an immobilized low-molecular-weight compound obtainable by the method according to claim 19.

22. A method for detecting the binding activity of a test low-molecular-weight compound to a binding molecule of claim 21, the method comprising:
(1) contacting the test low-molecular-weight compound with the binding molecule to form a complex, wherein either the test low-molecular-weight compound or the low-molecular-weight compound immobilized in claim 18 has been immobilized, and when the low-molecular-weight compound immobilized in claim 18 has been immobilized, a complex of a test low-molecular-weight compound with a binding molecule will be formed by any of the following methods (a) to (c):
(a) contacting the low-molecular-weight compound immobilized in claim 18 with the binding molecule in the presence of the test low-molecular-weight compound,
(b) contacting the test low-molecular-weight compound with the binding molecule, and then with the low-molecular-weight compound immobilized in claim 18, and
(c) contacting the low-molecular-weight compound immobilized in claim 18 with the binding molecule, and then with the test low-molecular-weight compound, and
(2) measuring a volume change caused by the binding of an immobilized low-molecular-weight compound or immobilized low-molecular-weight compound which was immobilized in claim 18 to the binding molecule.

23. A method for screening a low-molecular-weight compound capable of binding to a binding molecule, the method comprising detecting the activity of binding of the low-molecular-weight compound to the binding molecule by the method according to claim 22, and selecting a compound having the binding activity to the binding molecule.

24. A compound obtainable by the method according to claim 23, and has a similar binding activity to the binding molecule as that of the low-molecular-weight compound immobilized in claim 18.

25. The method according to claim 1, wherein the low-molecular-weight compound is a vitamin D3 derivative which is immobilized on a carrier via a linker and the binding molecule is a vitamin D3 receptor.

26. The method according to claim 25, wherein multiple kinds of vitamin D3 derivatives are immobilized on the same carrier.

27. The method according to claim 11, wherein the low-molecular-weight compound is a vitamin D3 derivative which is immobilized on a carrier via a linker and the binding molecule is a vitamin D3 receptor.

28. The method according to claim 12, wherein the low-molecular-weight compound is a vitamin D3 derivative which is immobilized on a carrier via a linker and the binding molecule is a vitamin D3 receptor.

29. The method according to claim 13, wherein the low-molecular-weight compound is a vitamin D3 derivative which is immobilized on a carrier via a linker, the binding molecule is a vitamin D3 receptor, and in step (1) the immobilized vitamin D3 derivative is contacted with a vitamin D3 receptor and a test compound.

30. The method according to claim 25, wherein the volume change is measured by surface plasmon resonance.

31. The method according to claim 27, wherein the volume change is measured by surface plasmon resonance.

32. The method according to claim 28, wherein the volume change is measured by surface plasmon resonance.

33. The method according to claim 29, wherein the volume change is measured by surface plasmon resonance.

34. A method for screening an agonist or an antagonist of a vitamin D3 receptor, the method comprising measuring the binding activity of a test compound to the vitamin D3 receptor according to the method of claim 29, and selecting a compound having the binding activity to the vitamin D3 receptor.

35. A carrier comprising one or multiple kinds of vitamin D3 derivatives immobilized thereon, wherein the carrier is a surface plasmon resonance sensor chip.

36. A vitamin D3 receptor agonist or antagonist obtainable by the method according to claim 34.

37. A pharmaceutical composition comprising the vitamin D3 receptor agonist or antagonist of claim 36.
